(19) United States
(12) Patent Application Publication
(10) Pub. No.: US 2025/0381445 A1
(43) Pub. Date: Dec. 18, 2025

(54) CUSTOM MOVEMENT PROGRAM AND ANALYTICAL FEEDBACK GENERATION

(71) Applicants: William Yuen, Miami Beach, FL (US); Jack R. Yuen, Miami Beach, FL (US)

(72) Inventors: William Yuen, Miami Beach, FL (US); Jack R. Yuen, Miami Beach, FL (US)

(21) Appl. No.: 19/297,849

(22) Filed: Aug. 12, 2025

Related U.S. Application Data
(63) Continuation-in-part of application No. 18/739,465, filed on Jun. 11, 2024, now Pat. No. 12,383,790.
Application No. 18/739,465 is a continuation of application No. 17/675,518, filed on Feb. 18, 2022, now Pat. No. 12,029,941.
(60) Provisional application No. 63/153,831, filed on Feb. 25, 2021.

(51) Int. Cl. A63B 24/00; A63B 71/06

(52) U.S. Cl. CPC A63B 24/0006; A63B 71/0622; A63B 2024/0015; A63B 2220/34; A63B 2220/40; A63B 2220/836

(57) ABSTRACT

A system for optimizing mechanics and movements in remote or outpatient physical therapy, rehabilitation, sports training, and injury prevention that uses several inertia measurement units (IMUs) to measure a user's motion while performing an action. The IMUs can have additional sensors connected to improve the system's ability to detect flaws in the user's motion. Furthermore, the system uses machine learning to detect and determine flaw in a user's motion from the IMU data. The system may generate feedback to improve the user's motion based on the detected flaws. Different feedback communication may be provided based on the performance of the user after the feedback is provided.

### CROSS-REFERENCE TO RELATED APPLICATIONS

The present patent application is a continuation-in-part and claims the priority benefit of U.S. patent application Ser. No. 18/739,465 filed Jun. 11, 2024, now U.S. Pat. No. 12,383,790, which claims priority benefit of U.S. patent application Ser. No. 17/675,518 filed Feb. 18, 2022, now U.S. Pat. No. 12,029,941, which claims the priority benefit of U.S. provisional patent application No. 63/153,831 filed Feb. 25, 2021, the disclosures of which are incorporated by reference herein.

### BACKGROUND OF THE INVENTION

### 1. Field of the Invention

The present disclosure is generally related to real-time sensor-based monitoring devices and associated methods for tracking and analyzing the motion of the body without the need for a human. More specifically, the present disclosure is related to using a sensor package or a suite of sensors that can be placed on different parts of the body, monitoring the sensor data is monitored, and analyzing the sensor data so as to provide a user feedback regarding how to adjust their movements to achieve different results.

### 2. Description of the Related Art

Physical rehabilitation is a critical component of recovery following injury or surgery. Traditionally, rehabilitation is conducted in person through one-on-one sessions between patients and therapists, typically held at the therapist's office. This arrangement can often be inconvenient for patients and may result in canceled appointments. Moreover, even skilled therapists face challenges in accurately assessing whether a patient is performing exercises correctly. For example, in repetitive exercises, it can be difficult to recall which repetitions were performed properly and which were not. Describing precisely what a patient did right or wrong is often just as challenging. Some exercises require physical cues that are hard to verify visually. For instance, exercises involving the contraction and holding of specific muscles—such as the glutes or core—may be impossible to assess without physically touching the patient, which can be uncomfortable or inappropriate for some. In more complex movements that demand simultaneous muscle engagement and coordination, even the most experienced therapists may struggle to observe and evaluate all necessary details in real time.

Poor form and improper movement during physical therapy can lead not only to suboptimal outcomes but, in some cases, to further injury. In certain situations, patients may unintentionally harm themselves while performing exercises incorrectly. This risk is not limited to those in therapy—healthy individuals, such as workers lifting heavy objects, are also vulnerable to injury when using improper posture or technique. Without a trained therapist or knowledgeable observer to provide real-time feedback, individuals may remain unaware of the risks they're taking. By accurately analyzing and scientifically tracking a patient's movements, therapy sessions can be optimized to deliver maximum benefit. Moreover, this approach can help prevent injury in healthy individuals and reduce the likelihood of worsening existing injuries or causing new ones.

Using sensors in sports and health is well known in the industry. There are several fitness watches on the market for tracking user steps and heart rate, but these devices are limited in the amount of data they can provides and cannot really monitor a user's motion accurately. As such, current devices do not have the ability to monitor and analyze all the parameters of a user's real-time motion in enough detail to predict outcomes (e.g., success, failure, injury) and to make recommendations (e.g., as to adjustments). While some sensors or cameras may capture data and images, there are presently no systems available that can automatically capture and use such data and images to generate predictions and recommendations (particularly as to fine adjustments) across different physical activities

There is therefore a need in the art for improved systems and methods of real-time sensor-based monitoring and analysis of physical movement.

### SUMMARY OF THE CLAIMED INVENTION

Embodiments of the present invention may include a system for remote/out-patient physical rehabilitation or injury prevention comprising a database that stores information regarding a plurality of different activities, each activity associated with a set of measurements regarding a body part. The system may further comprise one or more sensors configured to attach to one or more locations on a body of a user. The system may further comprise a computing device configured to receive a plurality of measurements from the sensors during performance of an activity by the user, generate a custom machine learning model based on an activity performed by the user and one or more characteristics of the user, generate one or more feedback communications to present to the user using machine learning, wherein generating the feedback communications is based on an identified deviations between the plurality of measurements and a default set of measurements; and update the machine learning model with a subsequent measurements from the sensors during performance of the activity after the feedback communication is generated to generate a different type of feedback communication.

### BRIEF DESCRIPTION OF THE DRAWINGS

FIG. 1 illustrates an exemplary network environment in which a system for monitoring and analyzing physical movement may be implemented.

FIG. 2 illustrates an exemplary machine learning (ML) database for a given physical movement.

FIG. 3 illustrates an exemplary inertia measurement unit (IMU) database.

FIG. 4 illustrates an exemplary suggestion database.

FIG. 5 is a flowchart illustrating an exemplary method for data collection regarding physical movement.

FIG. 6 is a flowchart illustrating an exemplary method for analyzing physical movement data.

FIG. 7 is a flowchart illustrating an exemplary method for making learning-based suggestions regarding physical movement.

FIG. 8 is a flowchart illustrating an exemplary method for learning-based suggestion refinement.

FIG. 9 is a flowchart illustrating an exemplary method for monitoring and analyzing physical movement.

FIG. 10 is a flowchart illustrating an exemplary method for locating and polling movement sensors.

FIG. 11 is a flowchart illustrating an exemplary method for movement monitoring and analysis.

FIG. 12 is a flowchart illustrating an exemplary method for practice monitoring and analysis.

FIG. 13 is a flowchart illustrating an exemplary method for instruction management.

FIG. 14 is an example neural network architecture.

### DETAILED DESCRIPTION

Embodiments of the present invention may include a sensor package or a suite of sensors designed to fit in to a small form factor. Additional sensors connected to the package can be adapted to any or physical activity in which different parameters may be monitored. Any number of sensor packages could be placed at different points of the user's body depending on the sport and the movement that needs to be tracked. Additional sensors can be added or connected to the core sensor package based on the specific needs of the user and the recommended exercise. For example, full-body or multi-muscle group exercises may require multiple sensor packages—supplemented by additional sensors—to accurately analyze and predict the user's movements. A comprehensive setup might include sensors placed on the knees, waist, shoulders, hands, feet, head, and shoes. Sensor packages in the shoes and gloves may also incorporate pressure sensors to monitor force distribution and contact intensity, enabling a more detailed understanding of biomechanics and exercise performance.

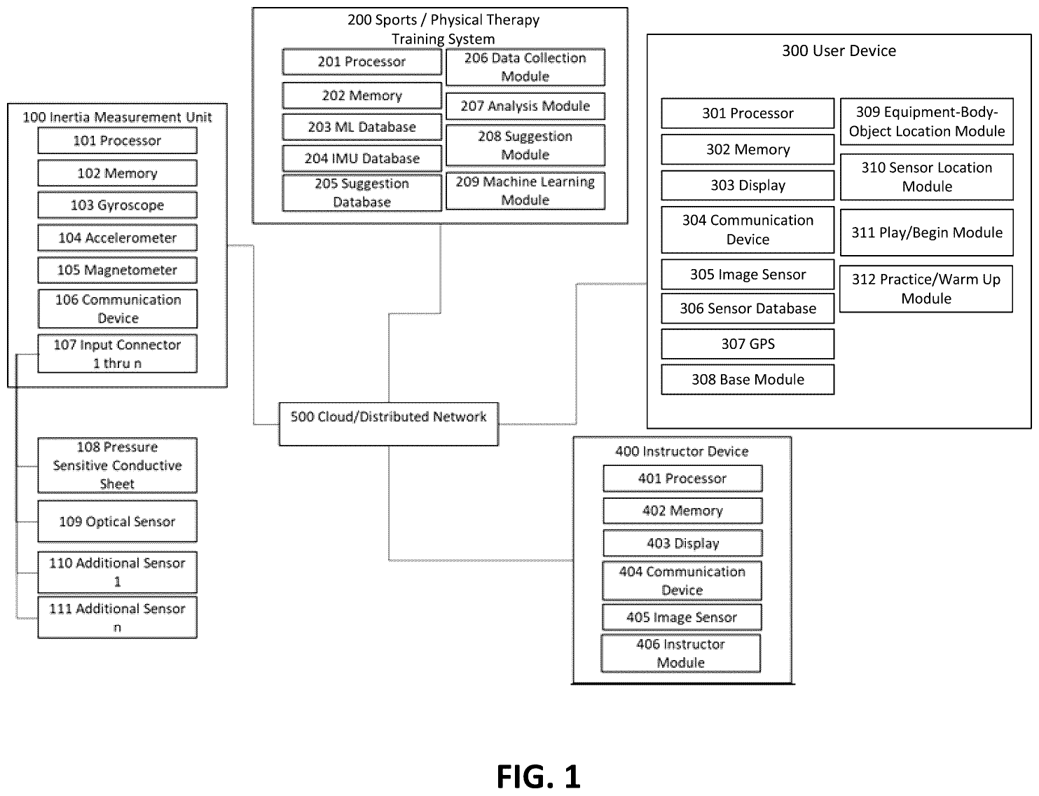

FIG. 1 illustrates an exemplary network environment in which a system for monitoring and analyzing physical movement may be implemented. The network environment of FIG. 1 may include an inertia measurement unit (IMU) 100. The IMU 100 is a suite of sensors designed to fit in a compact package which is then attached to a user, such as, a patient, or other individual, to monitor and analyze the motion of the wearer. The motion of the wearer may include walking, running, jumping, lifting, exercising, dancing, or performing athletic or other activity movements, such as swinging a golf club, swinging a bat, throwing a ball, hitting a ball, etc. The system may also analyze everyday movements for risk in relation to repetitive stress, strain, and other physical conditions. The system would further include several IMU 100 which a user would place on different parts of the body such as the arms, elbows, knees, waist, shoulders, head, feet, or hands. Further, the IMU 100 can also be placed within exercise or therapy equipment such as inside a floor mats, straps, training devices, tennis racquets, golf club head, shoes, or gloves (i.e. golf glove). Additionally, the IMU 100 allow additional sensors to be connected to adapt to different movements and movement-based activities (e.g., different sports, dances, exercises, yoga, physical therapies) and allow for the collection of other types of data such a pressure data. The IMU 100 may be inserted into every-day items such as shoes, socks, bracelets, anklets, or other worn accessories, watches, or clothing. For example, the IMU may be inserted into a shoe as a shoe insert to measure the magnitude and the direction of stresses or forces exerted by the user at different points within the foot (or other body part) relative to the ground, the distance the user has covered, the speed and the acceleration of the user over the period of time in which the movement is performed, etc. The IMU 100, further includes a processor 101, a memory 102, a gyroscope 103, an accelerometer 104, a magnetometer 105, a communication device 106, and any number of input connector 1 thru n 107. Further, other sensors or data collection devices may be connected to the input connectors 1 thru n 107 such as a pressure sensitive conductive sheet 108, an optical sensor 109, additional sensor 1 110 and additional sensor n 111.

A processor 101 may be used to execute an algorithms, code, or commands stored in the memory 102. The processor 101 may also be configured to decode and execute any instructions received from one or more other electronic devices, server(s), sensors, or other connected devices. The processor 101 may include one or more general purpose processors (e.g., INTEL® or Advanced Micro Devices® (AMD) microprocessors, ARM) and/or one or more special purpose processors (e.g., digital signal processors, Xilinx® System On Chip (SOC) Field Programmable Gate Array (FPGA) processor, and/or Graphics Processing Units (GPUs)). The processor 101 may be configured to execute one or more computer-readable program instructions, such as program instructions to carry out any of the functions described in this description.

The memory 102 is used to store information used in a computing device or related computer hardware such as the IMU 100. The memory 102 may be a semiconductor memory or metal-oxide-semiconductor (MOS) memory where data is stored within MOS memory cell. Examples of non-volatile memory are flash memory (used as secondary storage) and ROM, PROM, EPROM and EEPROM memory (used for storing firmware such as BIOS). Examples of volatile memory are primary storage, which is typically dynamic random-access memory (DRAM), and fast CPU cache memory, which is typically static random-access memory (SRAM) that is fast but energy-consuming, offering lower memory areal density than DRAM. The

Gyroscope 103 s a device used for measuring or maintaining orientation and angular velocity, such as the microchip-packaged MEMS gyroscopes found in electronic devices (sometimes called gyrometers). The accelerometer 104 is a device that measures proper acceleration. Proper acceleration is the acceleration (the rate of change of velocity) of a body. Two or more accelerometers 104 when coordinated with one another can measure differences in proper acceleration. The magnetometer 105 is a device that measures the direction, strength, or relative change of a magnetic field at a particular location. A compass is one such device, one that measures the direction of an ambient magnetic field, in this case, the Earth's magnetic field. The magnetometer 105 in the IMU 100 would provide directional data for any motion of a user. The communication device 106 is used for communicating data and commands to other IMU 100 or to other devices that are part of the system such as the sports/physical therapy training system 200, a user device 200 or an instructor device 400. The communication can be done though a wired connection or wirelessly user well know wireless communication devices and protocols such as Bluetooth, NFC, Wi-Fi standards, or cellular. Furthermore, the communication devices from at least two different IMUs 100 can be used to triangulate the location of a third IMU 100 by analyzing the communication signal. For example, an IMU 100 in both shoes of a user could be used in conjunction with a third IMU 100 embedded in a handheld implement—such as a golf club, rehabilitation aid, or physical therapy device—to determine the position and motion of the implement. This allows the system to track the relative positions of the user, the implement, and any relevant target (e.g., a golf ball or therapy marker), ensuring proper alignment, form, and technique whether in a sports/physical therapy training context or a therapeutic rehabilitation session. Based on the determined location, a recommendation can be provided to the user.

Input connectors 1 thru n 107 represent at least one means of connecting external devices such as other sensors to the IMU 100. This would allow the IMU 100 to be adapted to other physical or other movement-based activities by allowing additional sensors to be connected. The input connectors 1 thru n 107 may include, but not limited to USB, USB-C, thunderbolt, 4- 6- or 8 pin connectors. There are many other known connection devices that are well known in the art.

The pressure sensitive conductive sheet 108 is connected to the IMU 100 through the input connector 1 thru n 107. The pressure sensitive conductive sheet 108 is electrically conductive sheet that is flexible and can be incorporated into wearable items. For example, pressure sensitive sheets could be applied to or woven in to gloves or insoles of shoes. The sheets would be used to monitor pressure such as understanding a user's grip from a glove or tracking a user's weight distribution from the insole of the user's shoes For example, pressure-sensitive sheets could be applied to or woven into gloves or insoles of shoes. These sheets may be used to monitor pressure distribution, such as measuring grip force from a glove or tracking weight distribution from the insole of a user's shoes. In sports applications, for instance, the grip of a golfer on a club is highly specific and can significantly affect the accuracy of a swing-a pressure-sensitive sheet embedded in a golf glove could provide valuable data regarding grip technique. Similarly, in physical therapy settings, such sensors can help monitor patient progress by evaluating balance, gait, or hand strength during rehabilitation exercises, enabling more precise feedback and adjustment of therapy protocols. In another embodiment pressure sensitive sheet maybe in the insole of a user's shoe in other forms of clothing. The pressure sensitive conductive sheet 108 may be in the form of force plates. In an example, the force plate may be used to measure vertical jump performance by measuring the ground reaction forces during the jump. The pressure sensitive conductive sheet may be piezoelectric material or piezoresistive material. Such pressure sensitive conductive sheet 108 may aid in measuring jump height, peak or highest force exerted during the activity, change in momentum over time (impulse), power, the duration of time before the wearer leaves the ground, and the wearer's ability to quickly generate a force after a quick stretch (reactive strength index).

The optical sensor 109 such as an image sensor, CMOS, infrared sensor, or other types of optical sensor used for capturing images. The optical sensor would connect to the IMU 100 through the input connector 1 thru n 107. The optical sensor can be used for visual tracking motion. For example, an optical sensor on the brim of a hat that points towards a user's face could be used to track a user's eye movement. Alternatively, the optical sensor can be positioned to point directly forward—so that when a user is looking straight ahead, the sensor can capture whether and when an object, such as a club, bat, or rehabilitation tool, makes contact with a ball or target. It is often very difficult for an instructor or therapist to detect subtle eye movements or brief glances away from the intended focus. For example, if a user—whether an athlete during a swing or a patient performing a precision rehabilitation task—momentarily takes their eyes off the target, even for a fraction of a second, this lapse can significantly impact performance, accuracy, or therapeutic effectiveness. An optical sensor tracking eye focus can therefore provide valuable feedback in both athletic training and physical therapy contexts.

The additional sensor 110 and additional sensor n 111 represent any number of additional sensors that could be attached to the IMU 100 through the input connector 1 thru n 107. The additional sensors allow for the IMU 100 to be adapted to other types of sensors that can customize the IMU for different sports. For example, a swimmer may add different flow rate sensors or monitors to understand the flow of water over their body. In an embodiment, the sensor may be piezoelectric transducer or strain gauge transducer that measure the ground reaction force exerted by a wearer performing various activities, such as jumping.

Further, the sports/physical therapy training system 200 includes a processor 201, a memory 202, a ML database 203, a IMU database 204, a suggestion database 205, a data collection module 206, an analysis module 207, a suggestion module 208, and a machine learning module 209. The sport/physical therapy training system 200 collects data the user device 300 or directly from the IMUs 100. The data is collected and analyzed, then visualizations are developed depending on the activity engaged by the user or the injury of the user and sent back to both the user device 300 and the instructor device 400. The visualizations are then used by the system 200 to generate and provide feedback on how to improve the user's movement for a given activity in accordance with a defined or customized standard for a proper, successful, or otherwise preferred version of a movement. Physical therapists, instructors, trainers, or other professionals may also provide feedback that may be used to generate automated feedback to the user and other users exhibiting similar movement data. The sports/physical therapy training system 200 can further generate and provide automated feedback to a user based on real-time analysis of a movement during performance to help improve their performance by comparing IMU 100 data and associated analysis to similar historical analysis. In some instances, the sports/physical therapy training system 200 may detect signs of or predict a deviation from a standard for the given movement, so as to predict a need for feedback before the deviation occurs or proceeds. The feedback may include reminders on proper form and movement, warnings of potential deviation, improvement tips, tricks, exercises, current counts (e.g., for specific enumerated sets of movements), etc., to help improve abnormalities or deviations detected in the user's motion. The feedback may further include recommendations for movement to prevent injury. The sports/physical therapy training system 200 may communicate with the inertia measurement unit 100, the user device 300, and the instructor device 400 via cloud or distributed network 500. Depending on the user device and associated accessories and settings, the generated feedback may include any combination of audio, visual, audiovisual, textual, graphical, and/or haptic feedback, each of which may include or correspond to measurements, analyses, predictions, trends, etc., generated or tailored to the user and their movement(s).

The processor 201 may include one or more general purpose processors (e.g., INTEL® or Advanced Micro Devices® (AMD) microprocessors, ARM) and/or one or more special purpose processors (e.g., digital signal processors, Xilinx® System On Chip (SOC) Field Programmable Gate Array (FPGA) processor, and/or Graphics Processing Units (GPUs)). The processor 201 may be configured to execute one or more computer-readable program instructions, such as program instructions to carry out any of the functions described in this description.

The memory 202 is used to store information used in a computing device or related computer hardware. The memory 202 may be a semiconductor memory or metal-oxide-semiconductor (MOS) memory where data is stored within MOS memory cell. Examples of non-volatile memory are flash memory (used as secondary storage) and ROM, PROM, EPROM and EEPROM memory (used for storing firmware such as BIOS). Examples of volatile memory are primary storage, which is typically dynamic random-access memory (DRAM), and fast CPU cache memory, which is typically static random-access memory (SRAM) that is fast but energy-consuming, offering lower memory areal density than DRAM.

The ML Database 203 stores historical or all known IMU data which the machine learning module uses to compare to determine potential issues, weaknesses, or flaws associated with a user's motion or technique while the user is performing an activity. For example, the ML database 203 may store all data for flaws in a user's motion or techniques and what the associated remedies would be to fix the flaw. The ML Database 203 may further store default or baseline data to compare the user's motion for the similar activity. The ML Database 203 may further store default or baseline data for a certain body type, limitations, and health concerns. If a user—such as a golfer—has too much weight on their back leg and is slicing the ball, the machine learning (ML) database may associate IMU 100 data with this imbalance and identify corresponding corrective measures, such as a specific adjustment, drill, or exercise to address the issue. In another example, an IMU 100 located on a glove can use gyroscopic data to detect whether a user is over-rotating or under-rotating their hands or wrists, which may cause a golf ball to slice or hook. Similarly, in physical therapy or rehabilitation scenarios, these sensors can track whether a patient is correctly performing a movement or compensating with improper body mechanics. IMU data may further include metrics such as jump height, peak or maximum force exerted during an activity, change in momentum over time (impulse), power output, ground contact time, and the user's ability to generate force rapidly after a brief stretch (reactive strength index). These insights can be applied to optimize athletic performance, improve movement efficiency, or guide therapeutic interventions. IMU data may further include jump height, peak or highest force exerted during the activity, change in moment um over time (impulse), power, the duration of time before the wearer leaves the ground, and the wearer's ability to quickly generate a force after a quick stretch (reactive strength index). The IMU data may further include data of the user's motion or technique over time.

The IMU Database 204 stores all the IMU data received from either the user device 300 or the IMU 100. The stored data would include any sensor data, time stamps, user information, type of activity the user is using the IMU 100 for. The suggestion database 205 stores suggested lessons, tips, tricks, corrections, and exercises to help correct a flaw or correction in a motion of a user (i.e. athlete) or prevent injuries. The suggestion data may come in the form of text, animation, or videos.

The data collection module 206 communicates with the user device 300 and the IMUs 100 to collect and organize the data in the IMU Database 204. The analysis module 207 uses data stored in the IMU Database 204 and organized and normalizes the data based on the activity the IMUs 100 are being used for. It compares the IMU 100 data in the IMU Database 204 with data from what would be an ideal swing in golf or proper form in physical therapy. The IMU 100 data from the IMU Database 204 can then be mapped and charts and visualizations created to show how the IMU data compares to normal or accurate motion. For example, IMU data from for a user's golf swing would be compared to an ideal or perfect golf swing. The IMU 100 data of the user may be compared to the data of other users performing similar activity, having a similar body proportions, body type, injury, etc. The ideal or perfect golf swing may be different for each user as every user has differences. The comparison will show where the user's IMU data (i.e. motion data) falls outside the normal ranges for an ideal or perfect swing. These differences in ranges from the IMU data and the ideal or perfect swing can then be mapped to visualizations. For example, a heat map could be created of the user's motion and animated. The heat map could be a 3D representation of the user's body and shows an animation of the user's swing and body position. The IMU data is then mapped to the 3D representation for the entirety of the swing. If the IMU data is outside certain ranges, it would display red on the 3D representation. This way a user or an instructor could see the issues related to the user's swing through the whole animation of the swing. Other charts and graphs could also be generated comparing the IMU data to the ideal or perfect swing. In another embodiment, the swing which the user is compared to maybe a swing or style that the user has selected. For example, if a user wants to swing like a certain professional golfer, they could select that swing, and their swing would be compared to that professional golfer's swing. Furthermore, this could be applied to any other activities, including, but not limited to, baseball, football, swimming, jumping, lifting, running, walking, physical therapy exercises, etc.

Further, the suggestion module 208 works in conjunction with the machine learning module 209 to provide personalized feedback, including tips, drills, or exercises aimed at improving a user's movement or performance. For example, in sports settings, the system may offer swing corrections, while in physical therapy contexts, it may recommend rehabilitative exercises or adjustments to movement patterns. Data may be collected over time to track the user's progress and adapt future suggestions based on improvements or persistent deviations from optimal motion. Further, the suggestion module 208 works with machine learning module 209 to provide learning tips, trick, or exercises to improve a user's swing. The data may be collected at different time periods to track the progress of the user.

The machine learning module 209 may generate a machine learning model to determine the type of activity the user is engaged in by the data received from the sensors. For example, the machine learning module 209 may determine that the user is engaged in countermovement jump, a jump performed with a quick, controlled squat followed by a jump, by analyzing the user's movement, acceleration, and the detected pressure in sensors, etc. as opposed to a jump from a static squat position (squat jump) or a jump performed from a standing position (vertical jump). The machine learning module 209 may generate a custom machine learning model based on the specific exercise, training, physical therapy programs.

The machine learning module 209 may identify the characteristics of the user based on the body type, limitations, and health concerns of the user from the data of the user, such as height, weight, body shape, known injury, deformation, body proportions. The machine learning module 209 may gather and aggregate data of other users performing similar activity for a similar body type, similar injury, or limitations. The machine learning module 209 may generate a custom machine learning model based on the characteristics of the user for the specific exercise, training, physical therapy programs.

The machine learning module 209 may generate a machine learning model to determine where the problem areas are in the activity and to find tips, trick, lessons, or exercises the user can do to improve the motion. The improvement material may be stored in the suggestion database 205 where the improvement material (i.e. tips, tricks, lessons, or exercises) are associated with known problems or issues with a user's motion. For example, if it is identified that a user is placing excessive weight on their back leg during a specific movement—such as a golf swing or a therapeutic exercise—the suggestion module may recommend tips, corrective cues, or targeted exercises that are known to address and improve the identified issue. The machine learning module 209 may compare the IMU data of the user to stored data of similar activity or similar body type to determine how closely the user's motion follows the ideal motion or techniques. The machine learning module 209 may identify deviation in the user's measurements from the ideal measurements to determine weaknesses in jumping technique, such as imbalances in force production or poor landing mechanics.

The machine learning module 209 may automatically provide feedback or recommendation to improve or correct the identified problems and weaknesses based on the compared data. The recommendation may include identifying current problems, potential problems, and ways to reduce risk factors, the correct body position, timing, the correct force used and the involvement of the correct muscles to prevent injury, strengthen weakened areas, and to minimize future risks.

Further, the system may analyze the IMU data over time with the recommendations provided to the user to monitor the effectiveness of recommendations over time. The custom machine learning model generated by the machine learning module 209 may evolve over time with subsequent IMU data from the user, user's adherence to the program, and the user's progress. New IMU data received from the user after the recommendation is provided may be added to the machine learning model to improve the next recommendation provided to the user. For example, the machine learning model may generate a recommendation focusing on the areas of least improvement based on the historical data from the user. The system may determine the recommendation or feedback the user is most responsive to by tracking the user's improvement to the type of recommendation provided to produce the user's improvement. The user's improvement may be determined by the amount of positive change in the recent IMU data. The system may compare the amount of positive change over time in association with different types of recommendations to determine the most effective type of recommendation for the user. In another embodiment, the system may generate a different type of recommendation than the type of recommendation previously provided if the user does not show improvement or shows little improvement. For example, the recommendation may be updated to include a display comparing the user's movement to the default or ideal movement. In another example, the recommendation may shift the focus to a related body part different from the focus of the previously provided recommendation, such as swinging of the arm to generate more jump height rather than focusing on the legs. In another example, the recommendation may change to a different exercise to strengthen the same body part.

The machine learning model may evolve based on change in the user characteristics. The user may develop more muscles, lose weight, or heal from the injury. The machine learning model may be updated with the new user characteristics to provide a recommendation that fit the current characteristics of the user. For example, the recommendation may challenge the user further than previously provided to the user by changing the goals or providing different types of exercises. The machine learning module 209 may change the stored data that the user data is compared to based on the updated characteristics of the user to enhance the recommendation provided to the user.

In another embodiment, the suggestion module 208 is instructed by an instructor from the instructor device 400 on what material should be sent to the user and the user device 300 to help improve the user's motion. The machine learning module 209 compares user IMU data to other historical or known data to learn a user motion. For example, the system may use a user's IMU data to learn and analyze their movement patterns-such as a golf swing in a sports setting or a rehabilitation exercise in a physical therapy context. The machine learning module 209 then uses the comparison to suggest improvements based on known instruction related to user data (suggestion database). Furthermore, in another embodiment suggestions can be provided to the user base on user data such as user preference based on user input or questionnaire. For example, the user maybe asked during a set up process asks like height, weight, body type, age, specific injury/impairment to recommend a certain style of motion. For example, an individual's physical characteristics—such as height, body composition, or mobility limitations—may affect how they perform a given movement. A person with a larger midsection may not be able to, or may choose not to, perform a movement in the same way as someone with a taller or more athletic build.

Sensors placed on the body (e.g., at the waist, shoulders, or other key locations) can collect data to assess body shape, posture, and movement tendencies. Based on this information, the system can tailor movement recommendations—such as suggesting a suitable swing technique or therapeutic motion. The system may also reinforce proper performance by identifying successful outcomes (e.g., effective swings or correct therapeutic repetitions) and recommend adjustments when suboptimal performance is detected. The Machine learning module additionally monitors suggestions or feedback from an instructor and store that data in the ML Database for future reference. The system may analyze the IMU data over time to monitor the effectiveness of recommendations over time.

The user device 300 may comprise of a computer, tablet, or cellphone. These devices are well known in the art and would comprise of a processor 302, a memory 302, a display 303, a communication device 304, image sensor 305, sensor database 306, GPS 307, base module 308, equipment-body-object positioning location module 309, a sensor location module 310, a play module 311, and a practice module 312. The processor 301 may include one or more general purpose processors (e.g., INTEL® or Advanced Micro Devices® (AMD) microprocessors, ARM) and/or one or more special purpose processors (e.g., digital signal processors, Xilinx® System On Chip (SOC) Field Programmable Gate Array (FPGA) processor, and/or Graphics Processing Units (GPUs)). The processor 301 may be configured to execute one or more computer-readable program instructions, such as program instructions to carry out any of the functions described in this description. The memory 302 is used to store information used in a computing device or related computer hardware. The memory 302 may be a semiconductor memory or metal-oxide-semiconductor (MOS) memory where data is stored within MOS memory cell. Examples of non-volatile memory are flash memory (used as secondary storage) and ROM, PROM, EPROM and EEPROM memory (used for storing firmware such as BIOS). Examples of volatile memory are primary storage, which is typically dynamic random-access memory (DRAM), and fast CPU cache memory, which is typically static random-access memory (SRAM) that is fast but energy-consuming, offering lower memory areal density than DRAM. The display 303 is integrated into the user device 300 and will include a means for user input either through a touch element (i.e. touch display) or other input methods. The display may be a liquid crystal display (LCD), in-plane switching liquid crystal display (IPS-LCD), organic light-emitting diode (OLED), or active-matrix organic light-emitting diode (AMOLED). The communication device 304 is used for communicating data and commands to the IMU 100 or to other devices that are part of the system such as the sports/physical therapy training system 200, or an instructor device 400. The communication can be done though a wired connection or wirelessly via wireless communication devices and protocols such as Bluetooth, NFC, Wi-Fi standards, or cellular. The image sensor 305 such as a CMOS, infrared sensor, or other types of optical sensor used for capturing images. The image sensor 305 on the user device 300 can be used to capture images of the user's motions and used with the IMU data and the analysis module 207 to overlay the IMU data. For example, a user may use the user device to record their swing while using the training system. That image or video is captured and stored with the IMU data in the IMU Database 204. The sensor database 306 is like the IMU Database 204 located on the Sports/physical therapy training system 200 but is instead located on the user device 300. The sensor database 306 stores all the IMU 100 data as well as sensor data that may be collected from devices on the user device 300 such as the image sensor 305. In another embodiment the sound data captured from a microphone on the clothing of the user device 300 may also be stored and analyzed. Specifically, sound data may be used to analyze the acoustic characteristics of contact between two objects and determine whether the interaction was solid or optimal. For instance, in a sports setting, a clean strike—such as a golf club hitting a ball squarely—produces a distinct sound compared to an off-center hit. Similarly, in physical therapy applications, sound data can be used to assess the quality and consistency of repeated movements or interactions with therapeutic equipment, providing auditory feedback that helps evaluate performance and technique. In addition, sound sensors may be used to monitor breathwork or breathing techniques during physical activity or rehabilitation exercises, where controlled breathing is essential for performance, stability, or recovery. Irregular breathing patterns or poor breath control may be detected and addressed to improve overall movement quality and physiological efficiency. The GPS 307 or global positioning system can track the location of a user device 300. This data is used during the “play” function of a device. For example, when a user engages with the system during a physical activity—such as a round of golf or a therapeutic exercise—the system can not only track the user's motion using sensor data (e.g., swing or movement patterns), but can also use the location of the user's device to estimate outcome metrics, such as the distance and trajectory of an object impacted by the movement. In a sports context, this could include estimating how far a ball was hit and in what direction, while in a physical therapy setting, it could involve measuring range of motion, displacement of a limb or assistive device, or other spatial outcomes relevant to rehabilitation progress. This data can be used in conjunction with IMU 100 data from a handheld implement—such as a golf club, training aid, or therapeutic device—to analyze user-specific performance patterns. In a sports context, for example, it may help determine how far a user typically propels a ball or object using a specific club or tool, and whether the resulting motion followed an intended path or deviated (e.g., slicing or hooking). In physical therapy applications, similar analysis can be applied to assess movement symmetry, directional control, or consistency during rehabilitation exercises, enabling the system to detect deviations from optimal motion and provide corrective feedback.

The base module 308 initiates when the user opens the sports/physical therapy training or physical therapy application on the user device 300. The base module 308 will then initiate all other associated module in the user device. First the base module 308 checks to see if any new sensors are within communication of the user device or if there are any sensors in the database. Because the system is meant to be a modular system, any number of IMUs 100 can be added or removed from the system at any time. If a new sensor is detected or no sensors are registered int the senor database 306 the setup process begins and walks the user through a step-by-step process for adding or removing new IMUs 100. Once the setup is complete the user selects if they are practicing or playing. This will initiate the respective modules. The equipment-body-object positioning module 309 is initiated upon completion of the setup process and is triggered by the base module 308. The positioning module 309 uses communication devices 106 on IMUs 100 placed in both shoes of the user and on one or more pieces of equipment—such as a tennis racket, golf club, rehabilitation tool, article of clothing, accessory, or other worn or handheld item. This allows the system to determine the relative positions of different body parts and equipment in motion, enabling analysis of movement mechanics, object interaction (e.g., club-to-ball or hand-to-tool or arm-to-torso), and overall coordination in both athletic and therapeutic contexts. The module uses the communication devices 106 between at least three IMUs 100 to triangulate the locations of each. The use of triangulation using the signal from communication devices is well now in the art. For example, one possible method is to use the Bluetooth signal strength readings of three devices to calculate the position of one of the devices.

The sensor location module 310 is used during the setup phase of the system and is initiated by the play, begin or practice modules. The module is used to determine the location of the sensor based on the location of other sensors. The sensor location module 310 may use the same techniques described in the equipment-body-object location module 309. In another embodiment the location of the IMUs 100 can be generally determined by polling the sensor database 306. The general location of the sensors could be determined during the setup up process, for example, locations of the sensors may be determined based on the location on the body (i.e. elbow, knee, hand, etc.). Knowing the location of the sensors either by triangulation or generally based on the position on the body will allow for more accurate measurements when analyzing a user movement or motion data, such as a swing.

The play [or begin] module 311 is initiated if the user selects play [or begin] from the base module 308. The play [or begin] module 311 is used when a user wants to use the IMUs 100 and sports/physical therapy training/physical therapy system 200 while playing a game or performing a rehabilitation exercise. For example, if a user wanted to use the system to play a round of golf or begin a rehabilitation exercise, the play [or begin] module 311 would be initiated. The difference between using the IMUs 100 for play verses practice/warm up is that during a practice session/while warming up you may use all the possible IMUs 100 that are available. During a training session, warm-up, or guided rehabilitation exercise, a user may choose to wear IMUs 100 across multiple parts of the body for comprehensive motion tracking. However, during actual performance—such as active gameplay or the beginning of a rehabilitation routine—the user may prefer a less intrusive setup and opt to use only a limited set of sensors. For example, in a sports/physical therapy training context such as golf, the full system may include IMUs 100 placed in the club head, embedded in the user's shoes (e.g., insoles), and positioned around the body at key points such as the knees, waist, hips, shoulders, elbows, glove, or head. In contrast, during a live round of golf or similar activity, wearing all sensors may be cumbersome. In such cases, the play module 311 activates a streamlined configuration that leverages only the essential IMUs 100—such as those embedded in the equipment (e.g., club or training device), shoes, glove, or clothing—to continue tracking key motion and swing data without requiring full-body instrumentation. This enables the system to remain functional and informative while reducing user burden in both athletic and rehabilitative settings. Furthermore, the play/begin module 311 may utilize GPS 307 on the user device 300 to track the user's location throughout an activity. In a sports context such as golf, this functionality can be used to monitor the distance and trajectory of a ball or object following impact. Because IMUs 100 are embedded in the user's equipment—such as a golf club, training device, or rehabilitation tool—the system can identify which item is being used and evaluate the effectiveness or accuracy of the movement based on that context. For example, it can track shot accuracy relative to the specific club used in golf, or assess motion outcomes in physical therapy based on the tool or technique applied. This allows for performance tracking and progress evaluation across both athletic and therapeutic use cases. In another example, different jumps may be monitored. The system may determine that the user is engaged in countermovement jump, a jump performed with a quick, controlled squat followed by a jump, by analyzing the user's movement, acceleration, and the detected pressure in sensors, etc. as opposed to a jump from a static squat position (squat jump) or a jump performed from a standing position (vertical jump). The pressure sensitive conductive sheet 108, such as a force plate in the user's shoes may provide data regarding the pressure applied by the user and the pressure received during landing. The IMU data of the user's motion received from the sensors, such as force plates, may be tracked to determine how closely the user's motion follows the ideal motion or techniques. The system may analyze the data over time to monitor the effectiveness of recommendations over time.

The system may further use the equipment-object positioning module 309 in real-time or during active performance to provide the user with feedback on the correct positioning of an accessory or tool while performing the activity. This allows the system to guide proper form and alignment—such as ensuring correct placement of a sports implement during gameplay or proper positioning of a therapeutic device during rehabilitation exercises.

The practice/warm up module 312, as briefly described above would be initiated if a user wants to use the system during a practice or warm up session. During a practice/warm up session the practice module 312 would look for the maximum number of registered IMUs 100 that are registered or stored in the sensor data base 306. The practice module 306 may also initiate the image sensor 305 to be used to capture images of the user during their practice or warm up, for example while performing an exercise or practicing a golf swing. The play/begin module 311 and the practice module 312 are not limited to how may sensor each can user as described above. But in another embodiment the user would set up which sensor they would like to use during different situation in play or the beginning of a rehabilitation routine. One user might prefer more sensors while playing a round of golf or beginning a rehabilitation routine while a second user would prefer fewer.

The instructor device 400 may be a cell phone, tablet, computer, or similar device and include a processor 401, a memory 402, a display 403, a communication device 404, an image sensor 405, instructor module 406. Further, the instructor device 400 would be used by an instructor, therapist or a coach which allow them to review a patient's or an athlete's motion who is using the sports/physical therapy training/physical therapy system 200. The instructor device 400 allows the instructor, therapist or coach to view motion data and analysis as well as provide manual feedback to the user device 300. The processor 401 may include one or more general purpose processors (e.g., INTEL® or Advanced Micro Devices® (AMD) microprocessors, ARM) and/or one or more special purpose processors (e.g., digital signal processors, Xilinx® System On Chip (SOC) Field Programmable Gate Array (FPGA) processor, and/or Graphics Processing Units (GPUs)).

The processor 401 may be configured to execute one or more computer-readable program instructions, such as program instructions to carry out any of the functions described in this description. The memory 402 is used to store information used in a computing device or related computer hardware. The memory 402 may be a semiconductor memory or metal-oxide-semiconductor (MOS) memory where data is stored within MOS memory cell. Examples of non-volatile memory are flash memory (used as secondary storage) and ROM, PROM, EPROM and EEPROM memory (used for storing firmware such as BIOS). Examples of volatile memory are primary storage, which is typically dynamic random-access memory (DRAM), and fast CPU cache memory, which is typically static random-access memory (SRAM) that is fast but energy-consuming, offering lower memory areal density than DRAM. The display 403 is integrated into the instructor device 400 and will include a means for user input either through a touch element (i.e. touch display) or other input methods. The display may be a liquid crystal display (LCD), in-plane switching liquid crystal display (IPS-LCD), organic light-emitting diode (OLED), or active-matrix organic light-emitting diode (AMOLED). The communication device 404 is used for communicating data and commands to and from the user device 300 and the sports/physical therapy training system 200. The communication can be done though a wired connection or wirelessly user well know wireless communication devices and protocols such as Bluetooth, NFC, Wi-Fi standards, or cellular. The image sensor 405 such as a CMOS, infrared sensor, or other types of optical sensor used for capturing images. The image sensor 405 on the instructor device 400 can be used to capture images of the user's motions and used with the IMU data and the analysis module 407 to overlay the IMU data.

The instructor module 406 is initiated by the instructor or coach and would allow the instructor to see the user's data and analysis. When initiated the instructor module 406 will communicate with the user device 300 and the sports/physical therapy training system 200 and receive the user's IMU data and analysis. The motion analysis it displayed to the instructor, therapist or coach along with the suggestions or feedback that would be provided to the user. The instructor or coach can then provide their own feedback by selecting from the suggestion database 205 or recommending suggestions provided by the suggestion module 208. In another embodiment, the coach's feedback may be customized. The instructor, therapist or coach may provide other tips or tricks not in the suggestion database.

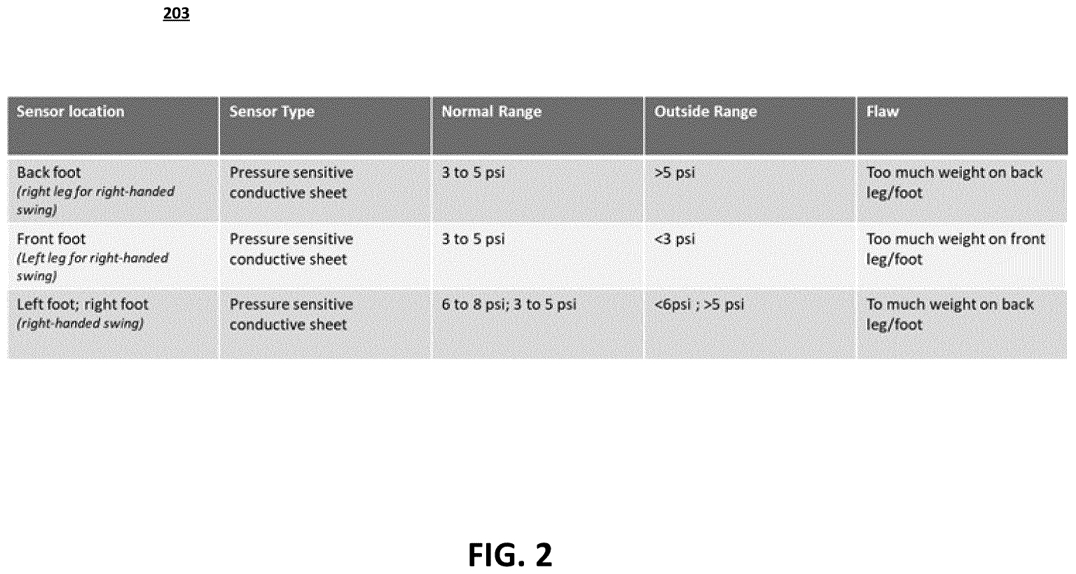

FIG. 2 illustrates an exemplary machine learning (ML) database 203 for a given physical movement. The ML database 203 stores historical or all known IMU data which the machine learning module uses to compare to determine potential issues, weaknesses, or flaws with a user's motion. For example, the machine learning (ML) database 203 may store example data representing common movement flaws—such as errors in a golfer's swing or improper form during a patient's rehabilitation exercise—along with the corresponding corrective actions or recommended interventions. For instance, if a user is placing too much weight on their back leg during a movement—resulting in a sliced golf shot or an unbalanced therapeutic motion—the ML database may recognize the associated IMU 100 data pattern and suggest an appropriate correction, such as a specific tip, cue, or targeted exercise to address and improve the issue. Specifically, for the above-mentioned example, the ML database 203 would store data related to how much pressure should be measured on a pressure sensitive conductive sheet 108 in the sole of a user's shoe. This pressure measurement may be a range. If measured data from the IMU 100 is outside that range it can be determined there is a flaw. If the measured pressure is too high and outside the range it would suggest to much weight on the back leg of the user. The example above considers only one sensor to identify a problem, but in another embodiment a group of sensor data can be used to identify a flaw in the user's motion.

In another example, the ML database 203 stores IMU data for a jumping technique to identify weaknesses in the technique, such as imbalances in force production or poor landing mechanics.

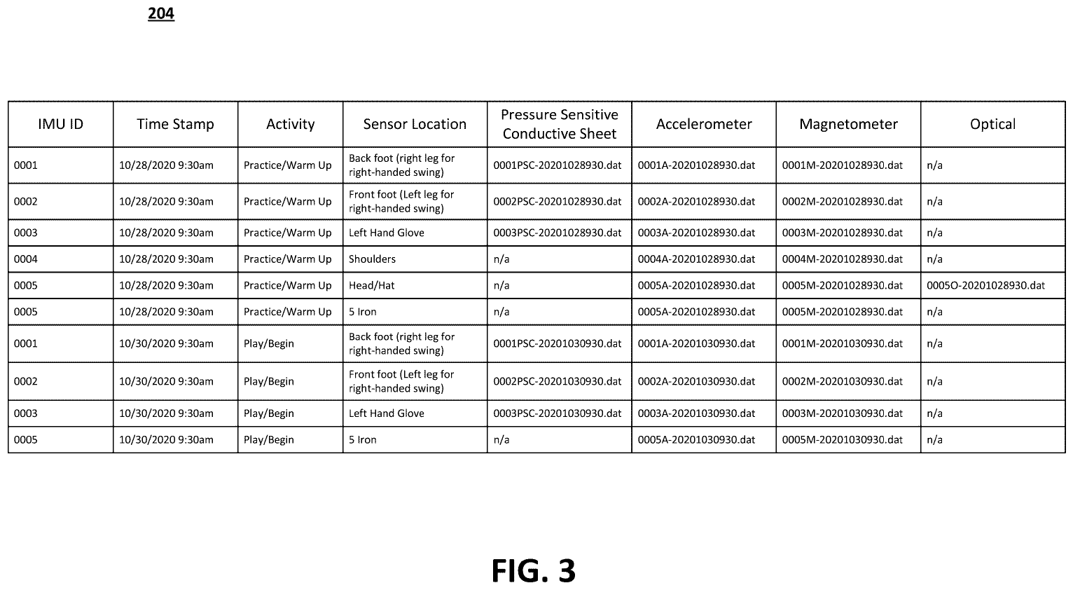

FIG. 3 illustrates an exemplary inertia measurement unit (IMU) database 204. The IMU database 204 stores all the IMU data received from either the user device 300 or the IMU 100. The stored data would include any sensor data, time stamps, user information, type of activity the user is using the IMU 100 for.

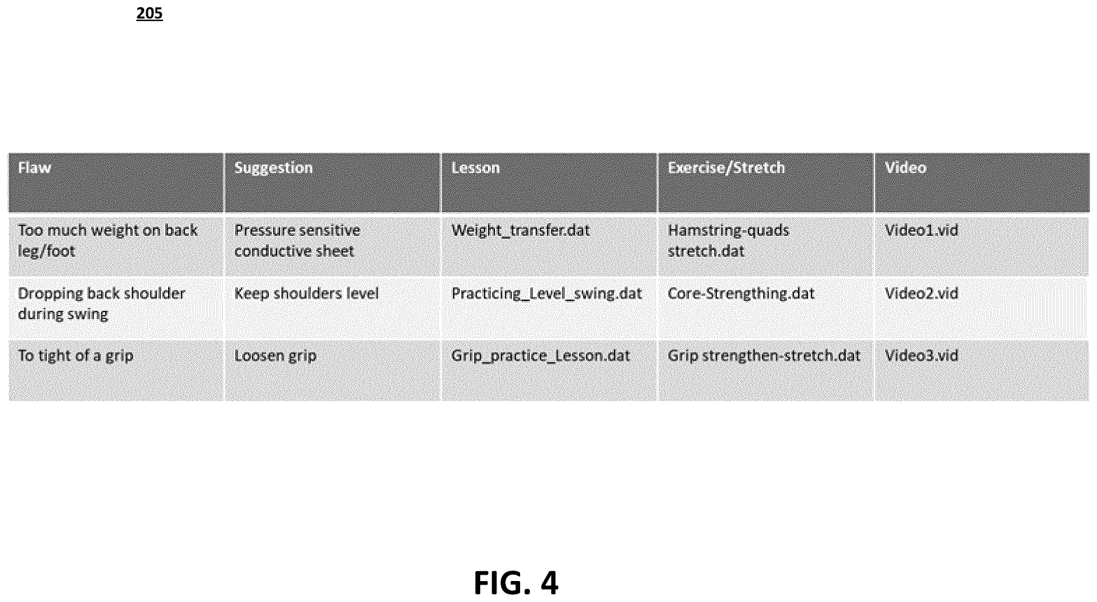

FIG. 4 illustrates an exemplary suggestion database 205. The suggestion database 205 stores suggested lessons, tips, tricks, corrections, and exercises to help correct a flaw or correction in a motion of a user (i.e. athlete or patient). The suggestion data may come in the form of text, animation, or videos. For example, if data from the IMUs 100 determines that pressure on the back foot of a user at the end of their swing is higher than normal ranges it may be determined that they are leaving to much weight on their back leg during their swing or exercise. The suggestion database 205 has any number of tips, tricks, exercises, or lessons for correcting the issues. For example, there may be a video the user can watch of an instructor, therapist or coach providing tips on how to correct the issue.

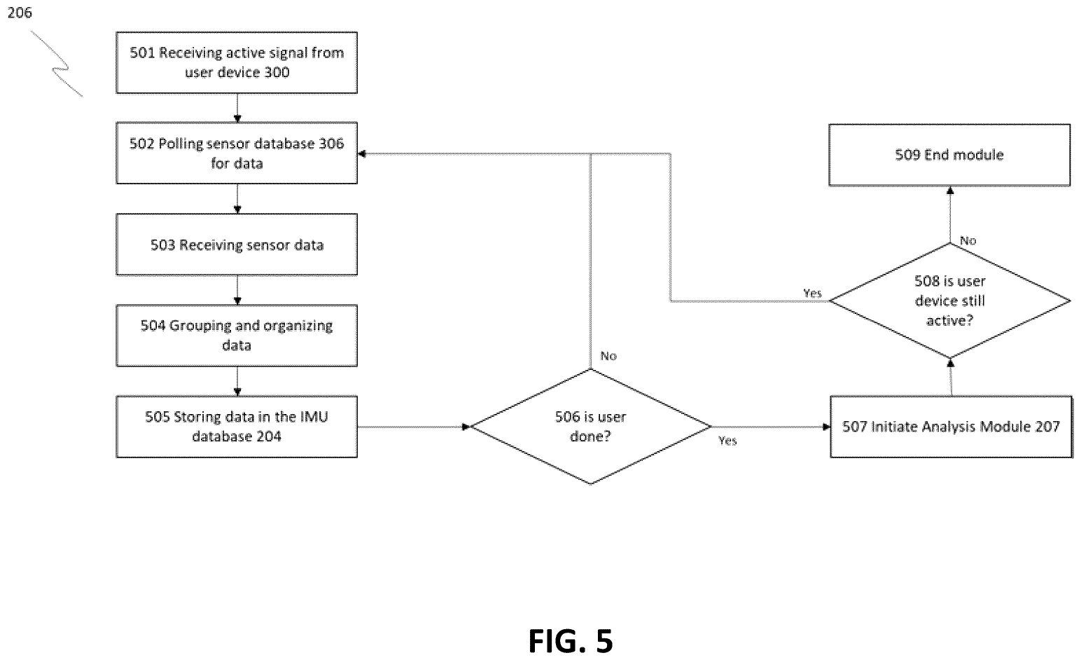

FIG. 5 is a flowchart illustrating an exemplary method for data collection regarding physical movement in accordance with execution of a data collection module 206. The data collection module 206 communicates with the user device 300 and the IMUs 100 to collect and organize the data in the IMU Database 204. The data collection module 206 begins with the module receiving a signal from the user device 300 that the user has initiated the system which will initiate the data collection module 206 at step 501. For example, this active signal may be generated when the user opens an app on the user device 300. Once the app loads it will connect with the sports/physical therapy training system 200 by sending the active signal which will allow the sports/physical therapy training system 200 to begin receiving data. Furthermore, the active signal can also be used for establishing a communications link directly between the user device 300 and the sports/physical therapy training system 200 for direct and secure data transfer. Once the data collection module 206 is active, the module begins to poll the sensor database 306 on the user device 300 for the most recent data, at step 502. The data is then received by the data collection module at step 503. The received data is then organized and grouped at step 504. The data that is being collected by the user device 300 from the IMUs 100 it is tracking the motion of user's movements such as a golf swing or a physical therapy exercise so data will be tracked over a period. There is also data coming from multiple IMUs 100 with any number of sensors. The data from multiple IMUs needs to be grouped and organize so that analysis can be performed accurately over a period such the time it takes to swing a golf club or perform a rehabilitation movement. The data is then stored in the IMU database 204 at step 505. The module then checks to see the user is still swinging or if they are done at step 506. If the user is not done and plans on collecting more data, the module returns to step 502. In another embodiment, the system and module would be able to determine when a user starts a specific motion such as a jump by knowing what activity the user is engaged in and by monitoring the IMU data. Data is then stored once it is determined that the user has started the motion, i.e. golf swing or rehabilitation movement. If the user is done with the motion, then the analysis module 207 is initiated at step 507. Once the analysis module is initiated at step 507 the module determines if the user device 300 is still active or if they user done at step 508. If the user is not done the module returns to step 502, otherwise the module ends at step 509.

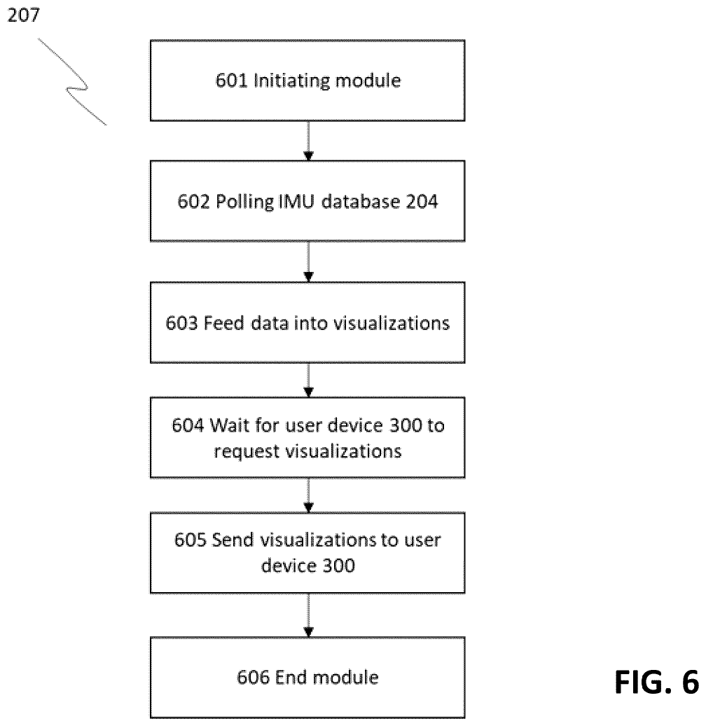

FIG. 6 is a flowchart illustrating an exemplary method for analyzing physical movement data in accordance with execution of analysis module 207. The analysis module 207 begins with the data collection module 207 initiating it after collecting and organizing all the data in to the IUM database 204, at step 601. The analysis module 207 then beings polling the IMU database for the organized data, at step 602. The data is then feed into models, chart, histograms, tables, and other visualizations at step 603. The visualizations may be pre-configured and just need data feed to them. Methods of using preconfigured visualizations and feeding data into them are well known in the art.

For example, IMU 100 data may be fed into a line graph to visualize pressure or weight distribution on a specific foot or body part during a given motion—such as a golf swing or a rehabilitation exercise. This allows the user to see how pressure or weight was distributed throughout the movement. The graph may also include reference ranges representing ideal or target distributions for the specific activity being performed. In one example, the line graph could incorporate data from a pressure-sensitive conductive sheet 108 embedded in the sole of the user's shoe—such as the back foot during a right-handed motion. The X-axis of the graph may represent time, spanning from the beginning to the end of the motion, while the Y-axis represents the amount of pressure applied. In a typical motion, such as a swing or squat, the graph may show even weight distribution at the start, followed by increased pressure on one side or limb (e.g., the back foot during a backswing or loading phase), and then a transfer of weight to the opposite side during the forward or exertion phase. Such visualization helps users, coaches, or therapists identify deviations from optimal weight transfer patterns and make informed adjustments to improve technique or therapeutic outcomes.

Another example of a visualization may include animations and 3D models. For instance, a 3D model of a human figure performing a specific motion—such as a golf swing or a rehabilitation exercise—could have data overlaid in the form of a heat map or other visual indicators. Sensor data (e.g., pressure, joint angles, or motion patterns) that falls within normal or optimal ranges may be displayed in green, while data points outside those ranges could appear in red or other contrasting colors. A user performing a motion with ideal form would see a 3D animated representation of their movement displaying green throughout, indicating proper technique. In both sports and physical therapy contexts, this type of visualization allows users, coaches, or therapists to identify areas needing improvement and reinforce correct movements with clear, real-time feedback. The module then waits for a request from the user device 300 at step 604. The data is stored in memory on the sports/physical therapy training system 200 until the user device 300 requests it as not to overwhelm the user device with too much data as it would have limited processing power. When a request for the visualizations is received from the user device 300, the visualizations are then sent to the user device at step 605. Once the visualizations are sent the module ends until initiated again by the data collection module 206 to create new visualizations, at step 606.

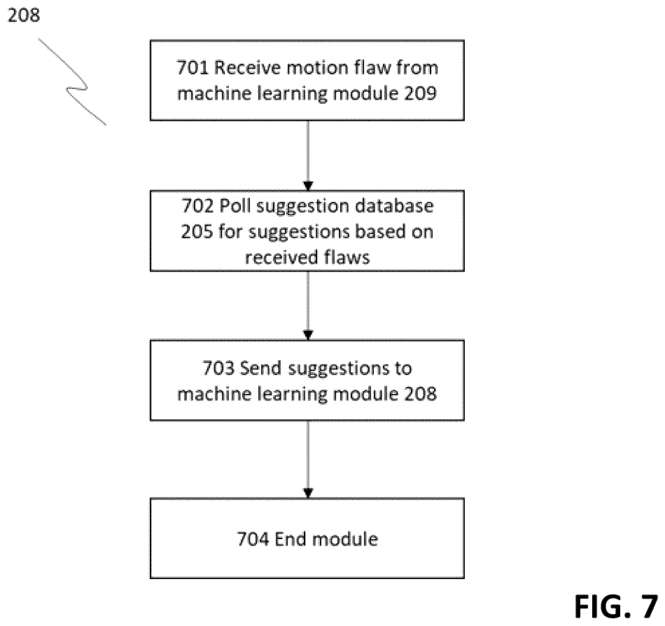

FIG. 7 is a flowchart illustrating an exemplary method for making learning-based suggestions regarding physical movement in accordance with execution of suggestion module 208. The suggestion module 208 begins with the receiving from the machine learning module 209 an identified flaw in a user's motion at step 701. For example, the machine learning module 209 may identify that the user has too much weight on their back foot on at the end of their swing in golf. Then the suggestion module 208 uses the received flaw to identify suggestions in the suggestion database 205, at step 702. There may be more than one suggestion for an identified flaw. Once flaws are identified they are sent back to the machine learning module 208 to be sent to the user device 300 and the instructor device 400.

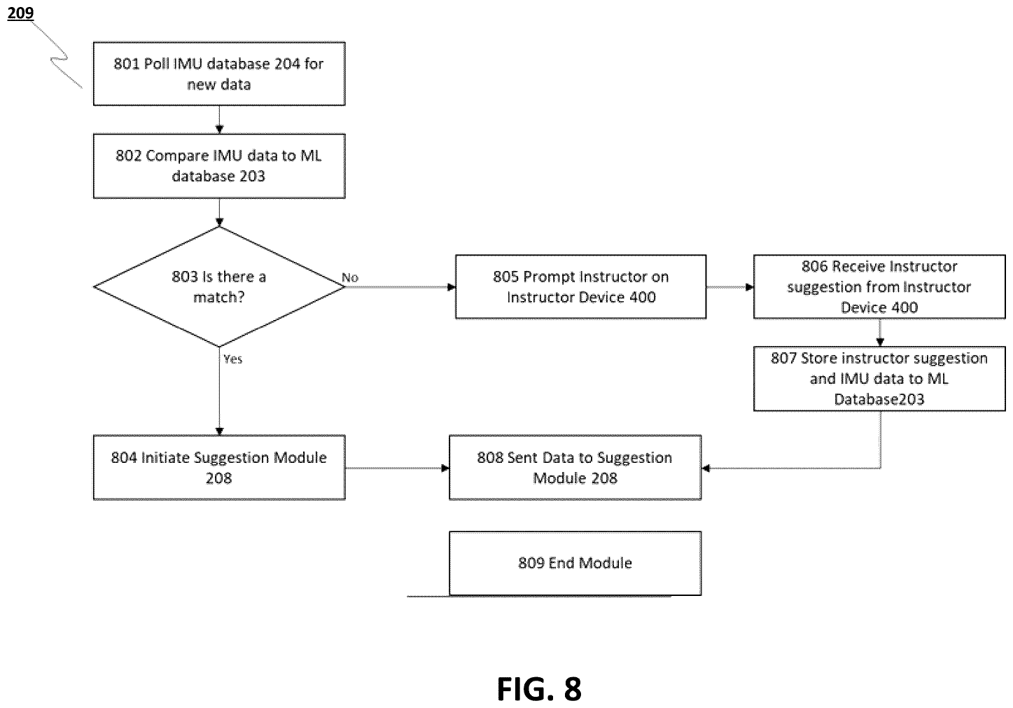

FIG. 8 is a flowchart illustrating an exemplary method for learning-based suggestion refinement in accordance with execution of a machine learning module 209. The machine learning module 209 begins with the polling of the IMU database 204 for the most recent or new IMU data, at step 801. The new or recent IMU data is then compared to data in the ML database 203, at step 802. The ML database 203 stores data the machine learning module 209 can use to identify specific flaws in a user's swing and then automatically send the flaw to the suggestion module 208 which will then send suggests to improve the user's motion or swing. If there is a match with the IMU data and the ML database 203 at step 803 then the suggestion module is initiated. If at step 803 there is no match, then the instructor is prompted on the instructor device 400 at step 805. The purpose of prompting the instructor is because the machine learning module 209 cannot figure out what the flaw is in the user's motion or swing or that the individual's body type, or has a physical limitation due to injury, is not built for a normal swing, so the data is sent to the instructor. The instructor reviews the IMU data from the user and determines what the flaw(s) is (are) [or the individual's special needs are in the user's motion or swing and the instructor sends the determined flaw back to the machine learning module 209 as step 806. The instructor's determined flaw is then stored into ML database 203 with the IMU data at step 807, to ensure that the next time when similar IMU data is received that machine learning module 209 will recognize the flaw. The determined flaw is then sent to the suggestion module 208, at step 808. The data sent to the suggestion module 208 may be the flaw determined and polled from the ML database 203 or may be the determined flaw from the instructor. Once the data is sent to the suggestion module the machine learning module 209 ends at step 809. In some embodiments when the IMU data is compared to the ML database 203, the data is matched based on a threshold comparison, specifically the IMU data may not match the data in the ML database 203 exactly but may match within a threshold.

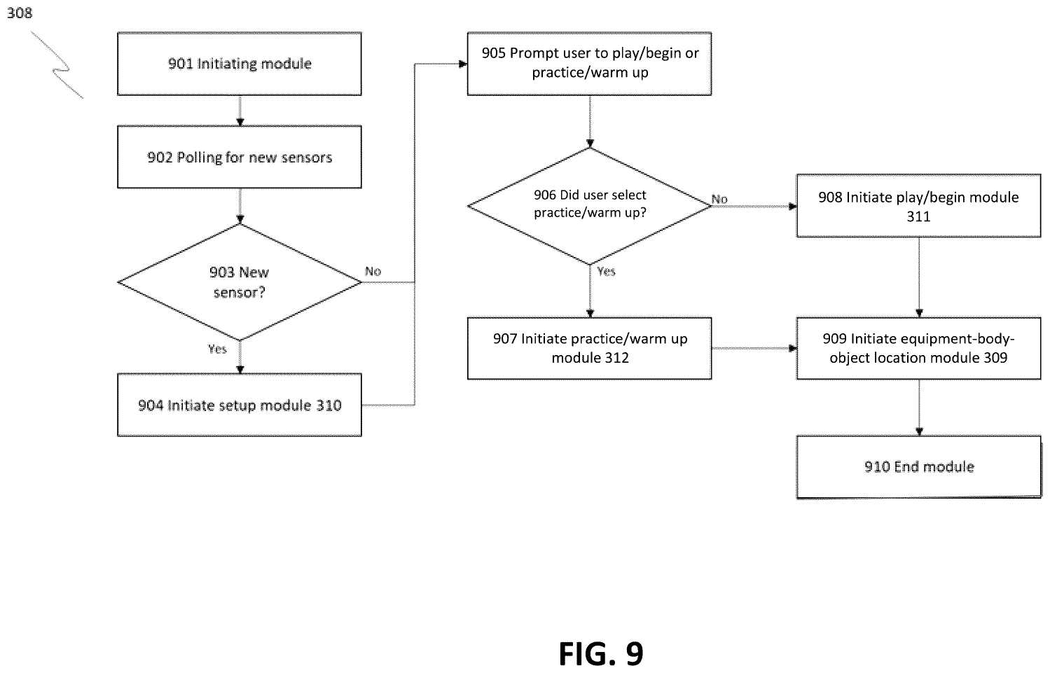

FIG. 9 is a flowchart illustrating an exemplary method for monitoring and analyzing physical movement in accordance with execution of base module 308. The base module 308 begins when the user initiates the application on their user device 300 which initiates the module at step 901. The base module 308 then begins to poll to see if there are new sensors that have been connected or within proximity of the user device at step 902. Specifically, if there are any additional IMUs 100 that are not already in the sensor database 306. The module will do this by looking at the current sensors connected to the user device 300 and the sensors currently stored in the sensor database 306. Sensors may be connected using known communication methods such as Bluetooth, NFC, or other methods of low power communications. If new sensors are detected, at step 903 the setup module 310 is then initiated at step 904. If no new sensors are detected at step 903 then the user is prompted if they are using the application to play or to practice at step 905. For example, when the golfer (patient) wants to use the application to practice their golf swing at the driving range (or warm up at home) they would select the practice/warm up option, while when the user wants to play a round of golf or begin the exercise they would select the play/begin option. The difference of the two options, play/begin vs. practice/warm up would determine which sensors the system would use. For example, for practice/warm up mode the system would not need to use the user device 300 GPS 307 but might leverage additional sensors when compared to when playing. At step 906 it is determined if the user selected “practice/warm up”, if yest then the practice/warm up module 312 is initiated at step 907. If the user selects “play/begin” instead of “practice/warm up” the play/begin module 311 is initiated at 908. Once the play/begin module 311 or the practice/warm up module 312 the equipment-body-object location module 309 is initiated at step 909. At this point all required modules for the user device 300 have been initiated and the module ends at 910.

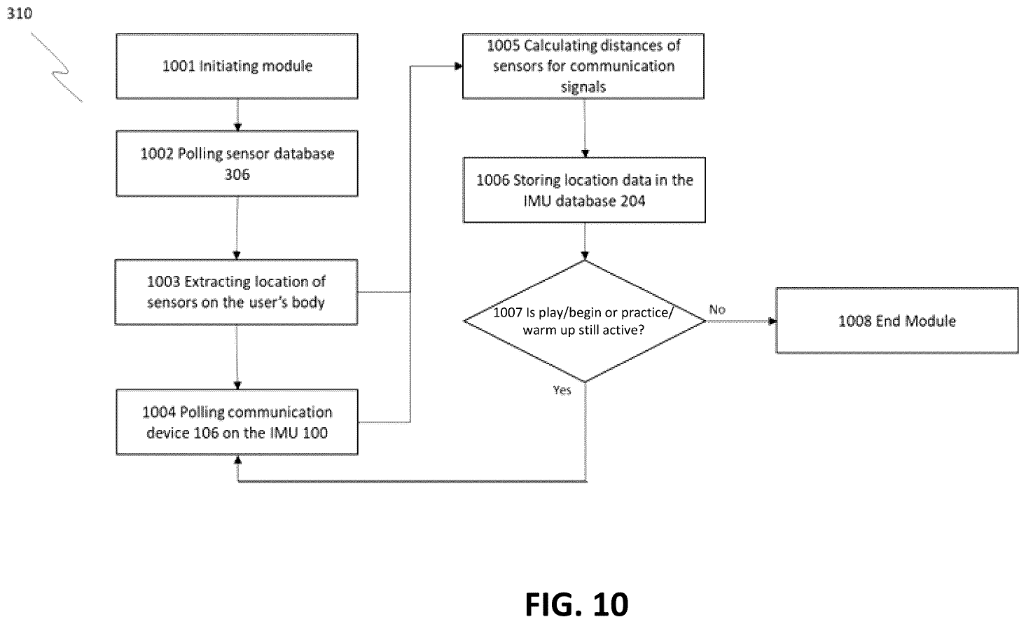

FIG. 10 is a flowchart illustrating an exemplary method for locating and polling movement sensors in accordance with execution of sensor location module 310. The sensor location module begins with the module being initiated by either the play/begin module 311 or the practice/warm up module 312 at step 1001. The sensor database 306 is then polled to determine how many and which sensors are already connected to the user device 300 as step 1002. The sensor database 306 stores the type of sensors or IMUs 100 that are connected to the system and the user device 300. Furthermore, the approximate location of the sensor on the user's body is also stored during setup process, for example, the location may be the foot, shoulders, club, or physical therapy equipment. The location of the sensors is extracted at step 1003. Once the general location on a user's body is determined the communication device on 106 on the IMU 100 is the polled for its signal data at step 1004. The signal data is then used to calculate the exact location of each sensor in relation to at least two other sensors at step 1005. Methods for calculating the exact location of a device using communications data is well known in the art. For example, leveraging the signal between three different devices, the signal strength can be used to calculate the location of each sensor from one another using triangulation. The location data is then stored in the IMU Database 204 at step 1006. The location data could also be stored locally in the sensor database 306 if there for some reason is not a connection to the sports/physical therapy training system 200. At step 1007 the module determines if the play/begin module 311 or practice/warm up module 312 is still active. If the play/begin module 311 or practice/warm up module 312 is still active the location data of all sensors is continuously calculated so the module returns to step 1004. The location of different sensors is needed to better understand the movement of a user's body. Furthermore, a sensor may be embedded in the head of a sports implement—such as a golf club or bat—or in a therapeutic device to help track the user's movement during an activity. This allows the system to capture detailed motion data, such as swing mechanics in sports or guided movement patterns in physical therapy, enabling accurate analysis and feedback for performance improvement or rehabilitation progress. If the play/begin module 311 or practice/warm up module 312 are no longer active the module ends at step 1008.

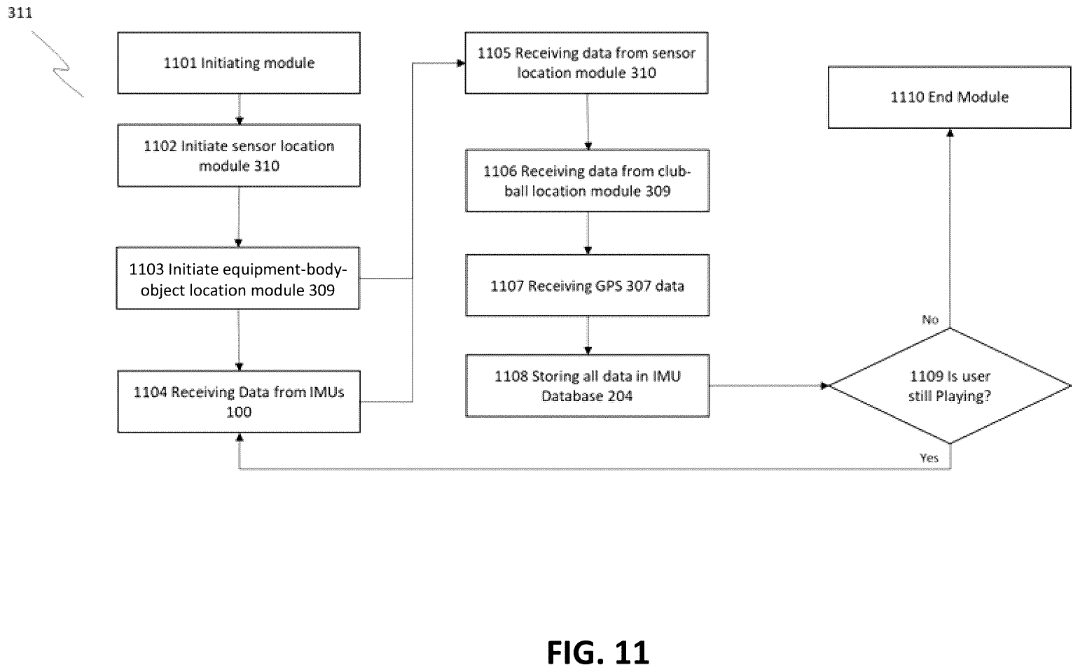

FIG. 11 is a flowchart illustrating an exemplary method for play/activity monitoring and analysis in accordance with execution of play/begin module 311. The play/begin module 311 begins with the base module 308 initiating the module when a user selects the option to “Play/Begin” at step 1101. The sensor location module 310 is then initiated at step 1102. This allows the play/begin module 311 to determine the exact location and which sensors are currently being used. For example, a user may choose to use only a limited number of sensors during an actual activity—such as playing a round of golf or performing a rehabilitation exercise—instead of utilizing all available sensors that might be worn during a dedicated practice or training session. In real-time scenarios, wearing a full set of sensors may be cumbersome or impractical. Therefore, the system can operate in a reduced mode, relying on strategically placed sensors—such as those embedded in equipment, footwear, or clothing—to continue tracking essential movement data while minimizing user burden. This approach enables the collection of meaningful performance or therapeutic data without compromising comfort or mobility—The reverse can also be the case. The equipment-body-object location module 309 is also initiated, which helps determine whether the positioning of relevant elements—such as the user's body, equipment, and any target object—is correct prior to initiating the activity, as shown at step 1103. For example, in a sports context such as golf, the module may assess whether the placement of the ball and club are properly aligned before a swing. In a physical therapy context, it may evaluate whether a therapeutic device or limb is correctly positioned before the user begins an exercise. This ensures proper setup for optimal performance, injury prevention, safety, and accuracy of motion tracking. Data is then received from the IMUs 100 that are connected and being used at step 1104. Data is also received from the sensor location module 310 at step 1105. Data is also received from the equipment-body-object location module 309 at step 1106. GPS data from the GPS 307 on the user device 300 is also receive at step 1107. GPS data can be used to track a user's performance and movements during play or therapy. For example, during an activity such as a round of golf or a mobility-based rehabilitation session, GPS data can be used to determine the outcome of a user's movement—such as estimating how far an object was propelled based on the user's location at the time of the motion. In a sports context, this may involve calculating the distance a ball was hit based on the user's position during the swing, while in a therapeutic setting, GPS or location data could be used to track walking distance, movement progression, or spatial accuracy during functional mobility exercises. All the sensor data including GPS data, IMU data, location data is then stored in the IMU database 306 at step 1108. Data could also be stored in the sensor database 306 before being sent to the sports/physical therapy training system 200 and the IMU database 204. It is then determined if the player is still playing/training or not at step 1109. If the user is still playing/training, then the system continues to receive data and returns to step 1104. If the user is no longer playing, then the module ends at step 1110.

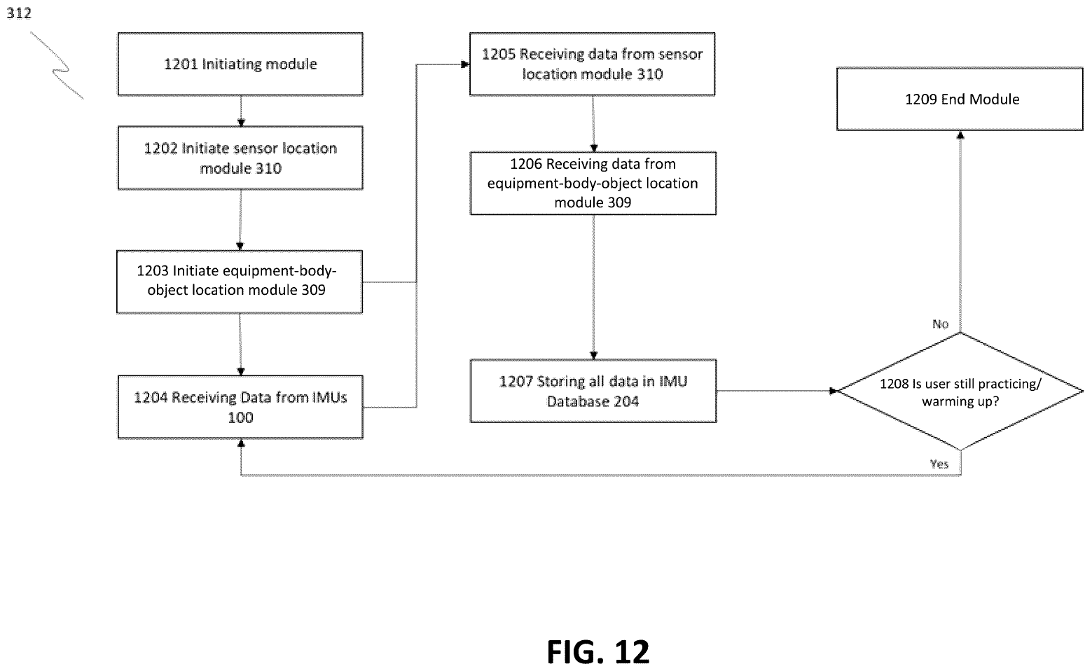

FIG. 12 is a flowchart illustrating an exemplary method for practice monitoring and analysis in accordance with execution of practice module 312. The practice module 312 begins with the base module 308 initiating the module when a user selects the option to “practice/warm up” at step 1201. The sensor location module 310 is then initiated at step 1202. This allows the practice/warm up module 312 to determine the exact location and which sensors are currently being used. For example, a user may choose to use all available sensors during a practice or training session to obtain a more detailed understanding and analysis of their motion—such as a swing in sports or a guided movement in physical therapy. The equipment-body-object location module 309 is also initiated, which assists in determining whether the positioning of relevant elements—such as the user's body, equipment, and any associated object—is correct prior to performing the activity, as shown at step 1203. In a sports scenario like golf, this may include verifying the alignment of the club and ball before a swing. In a therapeutic context, it may involve confirming the correct setup of a limb, device, or body position before beginning a prescribed exercise. Data is then received from the IMUs 100 that are connected and being used at step 1204. Data is also received from the sensor location module 310 at step 1205. Data is also received from the equipment-body-object location module 309 at step 1206. All the sensor data including IMU data, and sensor location data is then stored in the IMU database 306 at step 1207. Data could also be stored in the sensor database 306 before being sent to the sports/physical therapy training system 200 and the IMU database 204. It is then determined if the player is still practicing/warming up or not at step 1208. If the user is still practicing/warming up, then the system continues to receive data and returns to step 1204. If the user is no longer playing/exercising, then the module ends at step 1209.

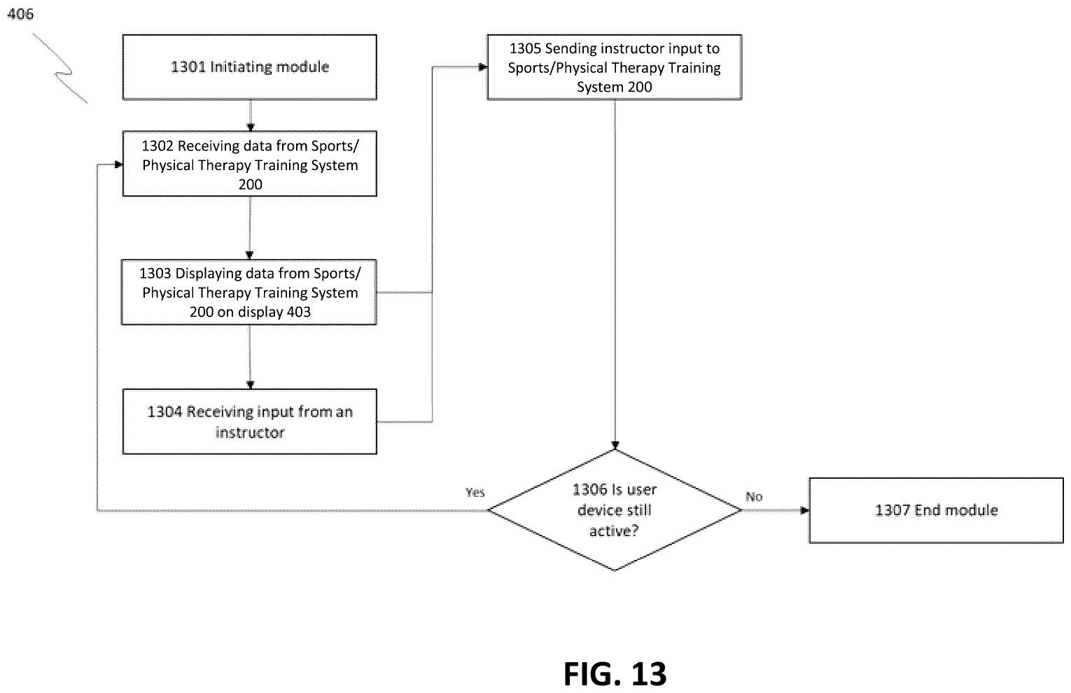

FIG. 13 is a flowchart illustrating an exemplary method for instruction management in accordance with execution of instructor module 406. The instructor module 406 begins with the module being initiated either by the instructor opening an application on the instructor device 400 or by a prompt from the sports/physical therapy training system 200 at step 1301. The prompt may come to the instructor device 400 in an alert or notification. For example, if the machine learning module 209 can determine a flaw while a user is practicing and the instructor isn't present it would prompt the instructor to determine the flaw to help further improve the machine learning algorithm and data. Once the instructor module 406 has been initiated the module receives data sent from the sports/physical therapy training system 200 at step 1302. The received data would be the IMU data from the user's IMUs 100. In another embodiment, the data received are recommendations from the machine learning module 209 on the closest matches to the IMU data for potential flaw in a user's motion. The received data is then displayed on the on the instructor device 400 display 403. The instructor would see analyzed data of the user's motion or swing and would be asked to provide feedback or to identify the flaw. The instructor or the machine learning model could provide suggested remedies, exercises, or lessons to improve the identified flaw or deviation. The instructor's input would then been received at step 1304. The instructor's input or the recommendation from the machine learning model would then be sent back to the sports/physical therapy training system 200 at step 1305. The instructor module 406 would then communicate with the user device 300 to determine if the user is still actively using the system at step 1306. If the user is still active the module would return to step 1302 to continue to receive data from the sports/physical therapy training system 200 and the instructor or the machine learning model would continue to provide feedback. Whereas if the user is no longer actively practicing or playing, then the instructors is no longer needed, and the module ends at step 1307. The IMU data received from the sensors, such as force plates, may be tracked over time and analyzed by the machine learning model to monitor the effectiveness of recommendations over time.

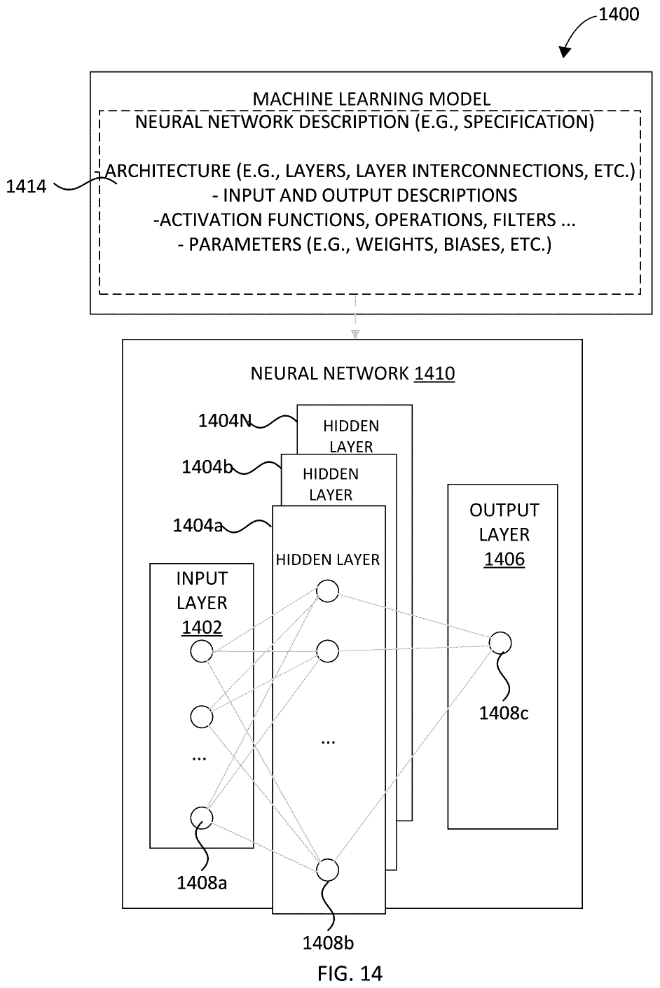

FIG. 14 illustrates an example neural network architecture that may be used to implement machine learning in relation to the AI-based processes described herein. Architecture 1400 includes a neural network 1410 defined by an example neural network description 1414 in node 1408c (neural controller). The neural network 1410 can represent a neural network implementation for machine learning module 209 of the sports/physical therapy training system 200. The neural network description 1414 can include a full specification of the neural network 1410, including the neural network architecture 1400. For example, the neural network description 1414 can include a description or specification of the architecture 1400 of the neural network 1410 (e.g., the layers, layer interconnections, number of nodes in each layer, etc.); an input and output description which indicates how the input and output are formed or processed; an indication of the activation functions in the neural network, the operations or filters in the neural network, etc.; neural network parameters such as weights, biases, etc.; and so forth.

The neural network 1410 reflects the architecture 1400 defined in the input layer 1402. In this example, the neural network 1410 includes an input layer 1402, which includes input data, such as stored historical data, stored default data, and input IMU data, user characteristics, feedback provided to the user.

The neural network 1410 further includes an output layer 1406 that provides an output (e.g., rendering output) resulting from the processing performed by the hidden layers 1404. In one illustrative example, the output layer 1406 can provide aggregated data of similar activities, similar types of users, and recommendations provided to the user. The neural network 1410 in this example is a multi-layer neural network of interconnected nodes. Each node can represent a piece of information. Information associated with the nodes is shared among the different layers and each layer retains information as information is processed. In some cases, the neural network 1410 can include a feed-forward neural network, in which case there are no feedback connections where outputs of the neural network are fed back into itself. In other cases, the neural network 1410 can include a recurrent neural network, which can have loops that allow information to be carried across nodes while reading in input. Information can be exchanged between nodes through node-to-node interconnections between the various layers.

Nodes of the input layer 1402 can activate a set of nodes in the first hidden layer 1404a. For example, as shown, each of the input nodes of the input layer 1402 is connected to each of the nodes of the first hidden layer 1404a. The nodes of the hidden layers hidden layer 1404a can transform the information of each input node by applying activation functions to the information. The information derived from the transformation can then be passed to and can activate the nodes of the next hidden layer (e.g., 1404b), which can perform their own designated functions. Example functions include convolutional, up-sampling, data transformation, pooling, and/or any other suitable functions. The output of the hidden layer (e.g., 1404b) can then activate nodes of the next hidden layer (e.g., 1404N), and so on. The output of the last hidden layer can activate one or more nodes of the output layer 1406, at which point an output is provided. In some cases, while nodes (e.g., nodes 1408a, 1408b, 1408c) in the neural network 1410 are shown as having multiple output lines, a node has a single output and all lines shown as being output from a node represent the same output value. In some cases, each node or interconnection between nodes can have a weight that is a set of parameters derived from training the neural network 1410.

For example, an interconnection between nodes can represent a piece of information learned about the interconnected nodes. The interconnection can have a numeric weight that can be tuned (e.g., based on a training dataset), allowing the neural network 1410 to be adaptive to inputs and able to learn as more data is processed. The neural network 1410 can be pre-trained to process the features from the data in the input layer 1402 using the different hidden layers 1404 in order to provide the output through the output layer 1406. In an example in which the neural network 1410 is used to identify the machine-learning factors, the neural network 1410 can be trained using training data that includes generated machine-learning factors, the stored current data, and the stored historical data including at least one of characteristics of the user, IMU data, data of similar users. For instance, training images can be input into the neural network 1410, which can be processed by the neural network 1410 to generate outputs which can be used to tune one or more aspects of the neural network 1410, such as weights, biases, etc. In some cases, the neural network 1410 can adjust weights of nodes using a training process called backpropagation. Backpropagation can include a forward pass, a loss function, a backward pass, and a weight update. The forward pass, loss function, backward pass, and parameter update is performed for one training iteration.

The process can be repeated for a certain number of iterations for each set of training data until the weights of the layers are accurately tuned. For a first training iteration for the neural network 1410, the output can include values that do not give preference to any particular class due to the weights being randomly selected at initialization. With the initial weights, the neural network 1410 is unable to determine low level features and thus cannot make an accurate determination of what the classification of the object might be. A loss function can be used to analyze errors in the output. Any suitable loss function definition can be used. The loss (or error) can be high for the first training dataset since the actual values will be different than the predicted output.

The goal of training is to minimize the amount of loss so that the predicted output comports with a target or ideal output. The neural network 1410 can perform a backward pass by determining which inputs (weights) most contributed to the loss of the neural network 1410, and can adjust the weights so that the loss decreases and is eventually minimized. A derivative of the loss with respect to the weights can be computed to determine the weights that contributed most to the loss of the neural network 1410. After the derivative is computed, a weight update can be performed by updating the weights of the filters. For example, the weights can be updated so that they change in the opposite direction of the gradient. A learning rate can be set to any suitable value, with a high learning rate including larger weight updates and a lower value indicating smaller weight updates.

The neural network 1410 can include any suitable neural or deep learning network. One example includes a convolutional neural network (CNN), which includes an input layer and an output layer, with multiple hidden layers between the input and out layers. The hidden layers of a CNN include a series of convolutional, nonlinear, pooling (for downsampling), and fully connected layers. In other examples, the neural network 1410 can represent any other neural or deep learning network, such as an autoencoder, a deep belief nets (DBNs), a recurrent neural networks (RNNs), etc.

For clarity of explanation, in some instances the present technology may be presented as including individual functional blocks including functional blocks comprising devices, device components, steps or routines in a method embodied in software, or combinations of hardware and software.

The functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

What is claimed is:

1. A system for injury prevention, the system comprising:
a database that stores information regarding a plurality of different activities, each activity associated with a set of movements defined by measurements taken regarding a body part over time;
one or more sensors configured to attach to one or more locations on a body of a user; and
a computing device configured to:
receive a plurality of measurements from the sensors during movement by the user;
generate a custom machine learning model based on an activity determined to be associated with the user movement and one or more characteristics of the user;
generate one or more feedback communications to present at a user device in accordance with the custom machine learning model, wherein generating the feedback communications is based on an identified deviations between the plurality of measurements and a default set of measurements; and
update the machine learning model with a subsequent measurements from the sensors during performance of the activity after the feedback communication is generated, wherein the updated custom machine learning model results in generation of a different type of feedback communication.

2. The system of claim 1, wherein the computing device is further configured to compare the plurality of measurements from the sensors with a plurality of measurements from other users performing a similar activity, wherein the feedback communications are further based on the comparison.

3. The system of claim 1, wherein the computing device is further configured to compare the plurality of measurements from the sensors with a plurality of measurements from other users with matching characteristics of the user, wherein the feedback communications are further based on the comparison.

4. The system of claim 1, wherein the characteristics of the user includes a type of injury.

5. The system of claim 1, wherein the characteristics of the user includes a body type.

6. The system of claim 1, wherein the computing device is further configured to determine a type of activity the user is performing based on the plurality of measurements.

7. The system of claim 1, wherein the one or more feedback communication is further based on an aggregate of the plurality of measurements from the sensors over time.

8. The system of claim 1, wherein generating the different type of feedback communication is based on determined amount of positive change over time in association with one type of feedback communication.

9. The system of claim 1, wherein the different type of feedback communication focuses on a different part of the body part than a body part in focus in the presented feedback communication.

10. The system of claim 1, wherein a different type of feedback communication is based on a change in the characteristics of the user.

11. A method for injury prevention, the method comprising:
storing in a database, information regarding a plurality of different activities, each activity associated with a set of movements defined by measurements taken regarding a body part over time;
attaching one or more sensors to one or more locations on a body of a user;
receiving a plurality of measurements from the sensors during movement by the user;
generating a custom machine learning model based on an activity determined to be associated with the user movement and one or more characteristics of the user;
generating one or more feedback communications to present at a user device in accordance with the custom machine learning model, wherein generating the feedback communications is based on an identified deviations between the plurality of measurements and a default set of measurements; and
updating the machine learning model with a subsequent measurements from the sensors during performance of the activity after the feedback communication is generated, wherein the updated custom machine learning model results in generation of a different type of feedback communication.

12. The method of claim 11, further comprising comparing the plurality of measurements from the sensors with a plurality of measurements from other users performing a similar activity, wherein the feedback communications are further based on the comparison.

13. The method of claim 11, further comprising comparing the plurality of measurements from the sensors with a plurality of measurements from other users with matching characteristics of the user, wherein the feedback communications are further based on the comparison.

14. The method of claim 11, wherein the characteristics of the user includes a type of injury.

15. The method of claim 11, wherein the characteristics of the user includes a body type.

16. The method of claim 11, wherein generating the custom machine learning model includes determining a type of activity the user is performing based on the plurality of measurements.

17. The method of claim 11, the one or more feedback communication is further based on an aggregate of the plurality of measurements from the sensors over time.

18. The method of claim 11, wherein generating the different type of feedback communication is based on determined amount of positive change over time in association with one type of feedback communication.

19. The method of claim 11, wherein a different type of feedback communication is based on a change in the characteristics of the user.

20. A non-transitory, computer-readable storage medium, having embodied thereon instructions executable to perform a method for injury prevention, the method comprising:
a database that stores information regarding a plurality of different activities, each activity associated with a set of movements defined by measurements taken regarding a body part over time;
one or more sensors configured to attach to one or more locations on a body of a user; and
a computing device configured to:
receive a plurality of measurements from the sensors during movement by the user;
generate a custom machine learning model based on an activity determined to be associated with the user movement and one or more characteristics of the user;
generate one or more feedback communications to present at a user device in accordance with the custom machine learning model, wherein generating the feedback communications is based on an identified deviations between the plurality of measurements and a default set of measurements; and
update the machine learning model with a subsequent measurements from the sensors during performance of the activity after the feedback communication is generated, wherein the updated custom machine learning model results in generation of a different type of feedback communication.
